(19) United States
(12) Patent Application Publication
(10) Pub. No.: US 2012/0219639 A1
(43) Pub. Date: Aug. 30, 2012

(54) NUTRITIONAL SUPPLEMENT FOR PATIENTS WITH CHRONIC HEART FAILURE

(75) Inventor: John Wigneswaran, Dayton, OH (US)

(73) Assignee: Nephroceuticals, LLC, Miamisburg, OH (US)

(21) Appl. No.: 13/468,110

(22) Filed: May 10, 2012

Related U.S. Application Data
(63) Continuation of application No. 12/631,332, filed on Dec. 4, 2009.

(51) Int. Cl. A61K 33/34; A61K 33/30; A61K 33/26; A61K 33/04; A23L 1/304; A61P 9/00; A61P 9/04; A23L 1/303; A23L 1/01; A61K 31/593; A61K 31/714

(52) U.S. Cl. 424/638; 514/168; 424/643; 424/646; 424/702; 514/52; 426/73; 426/2

(57) ABSTRACT

A nutritional supplement for patients having cardiovascular disease including heat failure. The nutritional supplement comprises vitamin C, vitamin D, vitamin B1, vitamin B2, vitamin B6, and folic acid, wherein the supplement is further characterized in that it does not contain vitamin A, vitamin K, or copper. In some embodiments, the supplement also contains omega fatty acids.

### CROSS-REFERENCE TO RELATED APPLICATION

This application is a continuation application of U.S. application Ser. No. 12/631,332 filed Dec. 4, 2009, and claims benefit thereto.

### FIELD OF THE INVENTION

The present invention is directed to a nutritional supplement for individuals suffering from cardiovascular disease (“CVD”) and more particularly, chronic heart failure (hereinafter “HF”). HF is a medical condition characterized by the inability of the heart to effectively supply organs with adequate blood and oxygen. People with HF have unique nutritional requirements as a result of alterations in absorption, metabolism, and excretion of many vitamins, minerals, and electrolytes due to factors related to the disease itself or its treatments (1). As a result, the intake of certain nutrients must be avoided or reduced, while higher doses of other nutrients are required to maintain health. Lastly, specific nutrients such as fish oil, not typically found in commonly available multivitamin preparations, are of importance to maintain health and are included in the present invention.

### DESCRIPTION OF THE INVENTION

The present invention provides a nutritional supplement for people with CVD and HF that in one embodiment comprises the following agents in the following approximate amounts:

| Vitamin/mineral | Range | |
|---|---|---|
| Vitamin C | 60-150 | |
| Vitamin D (as cholecalciferol) | 400-10,000 | IU |
| Vitamin E (as d-alpha tocopherol) | 0-400 | IU |
| Thiamin (Vitamin B1) | 1-200 | mg |
| Riboflavin (Vitamin B2) | 1.0-10 | mg |
| Niacin (as Niacinamide) | 0-1000 | mg |
| Vitamin B6 | 2-200 | mg |
| Folic Acid | 0.4-5 | mg |
| Vitamin B12 | 0-1000 | mcg |
| Zinc | 0-50 | mg |
| Selenium | 0-400 | mcg |
| Omega-3 Fatty Acids | 0-4000 | mg |

The supplement is also characterized by what it does not contain. Certain agents that are not desirable for CVD or HF patients when administered or consumed on a regular or a daily basis or may counteract the actions of concurrent medications that patients with HF take are not present. In one embodiment the supplement does not contain copper; and, in one embodiment it also does not contain Vitamin A. We are also not including Vitamin K as patients with CVD or HF often take warfarin for related medical conditions, and Vitamin K can antagonize the action of that medication resulting in harm. In addition, Vitamin E has been shown to increase the risk of heart failure (2) and all-cause mortality (3) when used in high doses, and therefore its use is limited to small amounts needed to preserve the fish oil. Copper levels have been noted to be increased in HF patients (4) and therefore in one embodiment copper is excluded as well. While it is desirable to eliminate the foregoing nutrients entirely, those skilled in the art will recognize that the addition of amounts of these excluded agents that are too small to harm the patient will not avoid infringement of this invention. While in one embodiment, the supplement contains no Vitamin A, up to about 0.7 mg of RE (retinol equivalents) may be tolerated and formulations including such amounts are not outside the scope of the invention. Vitamin E may be tolerated up to 100 IU/day, and copper may be tolerated up to 2 milligrams daily, and Vitamin K may be tolerated up to 80 mcg/day.

In a further embodiment, the supplement preferably includes specific vitamins and minerals and omega-3 fatty acids (DHA+EPA) from fish oil.

Vitamin D: Vitamin D deficiency is widespread in patients with CVD and HF for several reasons including reduced endogenous synthesis of D3 through reduced UVB exposure and lower dietary intake (5). Low Vitamin D has been shown to be involved in the process of atherosclerosis (6,7), and low vitamin D levels have been associated with increased severity of heart failure (8) and increased risk of death from heart failure (9). Results from a well established community based sample of patients (Framingham Heart Study) has shown that Vitamin D deficiency increased the risk of cardiovascular disease (which includes heart attack, stroke, peripheral arterial disease, or heart failure) especially in those with existing high blood pressure (10). Though the RDI of Vitamin D is currently 400 IU, a number of studies have shown that doses in excess of 800 IU correlate with reduced rates of fractures, falls, and cancer (11,12,13). In addition, a study of vitamin D with doses of 2000 IU have shown improvement in the cytokine profile in people with HF (14). A meta-analysis of clinical trials with over 57,000 patients showed that Vitamin D supplementation reduced the risk of death, in part by reducing cardiovascular-related deaths (15). In one embodiment, the supplement contains about 400 to 2000 IU, in another embodiment it contains about 1000 to 1500 IU to prevent deficiency based on the above guidelines and evidence.

Vitamin E: In one embodiment up to about 400 IU is included. In another embodiment, the supplement contains about 10 IU.

Vitamin C : Some studies in people with HF have shown decreased intake (8) of vitamin C Acute, high dose administration of Vitamin C improved baroreflex sensitivity in people with HF (9) and improved endothelial dysfunction (10). In one embodiment Vitamin C is limited to not more than 500 mg. The Tolerable upper limit for Vitamin C is 2000 mg. In one embodiment, the supplement contains no vitamin C. In another, embodiment, the supplement contains 500 mg. In another embodiment, the supplement contains 60-120 mg of Vitamin C.

Thiamine (vitamin B1), Riboflavin (Vitamin B2) and Pyridoxine (Vitamin B6): Numerous studies have shown deficiencies in thiamine, riboflavin, and pyridoxine in people with heart failure which may be related to medications used to treat heart failure (11,12). Though higher than the RDI, in accordance with the embodiment illustrated in the table doses of Vitamin B1 can run up to about 200 mg and doses of Vitamin B2 can run up to 10 mg. In a particular embodiment doses of vitamin B1 will be about 1 to 10 mg. In one embodiment, the dose of Vitamin B2 will be about 1 to 4 mg. In one embodiment, the dose of Vitamin B6 will be about 2 to 200 mg.

Zinc and Selenium: Deficiencies in zinc and selenium have been seen in patients with HF (13,14). This may occur because of the disease itself, or because of medications that are often administered to treat patients with HF. In addition, higher levels of selenium in the blood were associated with reduced incidence of coronary artery disease (23) while zinc deficiency was associated with higher mortality in people with coronary artery disease (24). In one embodiment, the dose of zinc will be about 8 mg to 50 mg. In one embodiment, the dose of selenium will be about 55 mcg to 400 mcg.

Omega-3 Fatty Acids (DHA+EPA): Omega-3 fatty acids, specifically docosahexaenoic acid (DHA) and eicosapentaenoic acid (EPA), have been shown in multiple prospective clinical trials and observational studies to significantly reduce the risk of cardiovascular morbidity and mortality (15). Omega-3 fatty acids have also been shown to decrease the risk death and hospitalization in patient with HF (16). Omega-3 fatty acids have also been reported to have beneficial effects on blood pressure and hyperlipidemia and may have anti-inflammatory properties that lead to a reduction in atherosclerosis. The American Heart Association's Scientific Statement has recommended intake of omega-3 fatty acids as part of a comprehensive strategy to prevent cardiovascular events, with about 1000 mg of omega-3 fatty acids suggested for secondary prevention of cardiovascular disease (27). While certain embodiments of the invention may employ up to 4000 mg of these fatty acids, in another embodiment, no fish oil is present. When present about 1000 mg appears sufficient, but the skilled artisan can readily increase this. In a particular embodiment about 850 to 1000 mg is used.

As described herein, by omitting certain nutrients, adjusting the doses of others, and adding nutrients typically not found in multivitamins—the formulations disclosed herein represents an approach to nutritional supplementation in HF.

The invention is illustrated by the following non-limiting examples.

### Example A

| Ingredient | Amount | |
|---|---|---|
| Folic Acid | 0.4 | mg |
| B6 | 5 | mg |
| B12 | 6 | mcg |
| Vitamin C | 60 | mg |
| Vitamin D (cholecalciferol) | 1000 | IU |
| Fish Oil (DHA + EPA) | 10000 | mg |
| Vitamin E | 10 | IU |
| Thiamine | 3 | mg |
| Riboflavin | 2 | mg |
| Niacin | 20 | mg |
| Zinc | 8 | mg |
| Selenium | 55 | mcg |

### Example B

| Ingredient | Amount | |
|---|---|---|
| Folic Acid | 0.8 | mg |
| B6 | 5 | mg |
| B12 | 6 | mcg |
| Vitamin C | 60 | mg |
| Vitamin D (cholecalciferol) | 1000 | IU |
| Fish Oil (DHA + EPA) | 850 | mg |
| Vitamin E | 10 | IU |
| Thiamine | 3 | mg |
| Riboflavin | 2 | mg |
| Niacin | 20 | mg |
| Zinc | 8 | mg |
| Selenium | 55 | mcg |

### Example C

| Ingredient | Amount | |
|---|---|---|
| Folic Acid | 1 | mg |
| B6 | 5 | mg |
| B12 | 2.4 | mcg |
| Iron | 8 mg (elemental) | |
| Vitamin C | 60 | mg |
| Vitamin D (cholecalciferol) | 1000 | IU |
| Vitamin E | 10 | IU |
| Thiamine | 3 | mg |
| Riboflavin | 2 | mg |
| Niacin | 20 | mg |
| Zinc | 8 | mg |
| Fish Oil (DHA + EPA) | 900 | mg |
| Selenium | 200 | mcg |

### Example D

| Ingredient | Amount | |
|---|---|---|
| Folic Acid | 0.8 | mg |
| B6 | 5 | mg |
| B12 | 2.4 | mcg |
| Vitamin C | 60 | mg |
| Vitamin D (cholecalciferol) | 1000 | IU |
| Vitamin E | 10 | IU |
| Thiamine | 3 | mg |
| Riboflavin | 2 | mg |
| Niacin | 20 | mg |
| Zinc | 8 | mg |
| Copper | 900 | mcg |
| Selenium | 55 | mcg |

References:
1. Kalantar-Zadeh, K. Am J Cardiol 2008; 101[suppl]:89e
2. Lonn, E. JAMA 2005 Mar. 16;293(11):1338-47.
3. Miller, E. Ann Intern Med. 2005;142:37-46.
4. Shokrzadeh, M. Biol Trace Elem Res 2009; 127(2):116
5. Kim, D. Am J Cardiol 2008;102:1540-1544.
6. Oh, J. Circulation. 2009;120:687-698.
7. Mitsuhashi, T. J Clin Invest. 1991;87:1889-1895
8. Zitterman, A. J Am Coll Cardiol 2003;41:105-12
9. Pilz, S. J Clin Endocrinol Metab 93: 3927-3935,2008
10. Wang, T. Circulation 2008: 117:503
11. Chapuy, M. N Engl J Med 1992; 327:1637
12. Bischoff-Ferrari, H. Am J Clin Nutr 2006;84:18-28.
13. Lappe, J. Am J Clin Nut 2007; 85(6):1586
14. Schleitoff, S. Am J Clin Nutr 2006;83:754-9
15. Autier, P. Arch Int Med 2007; 167:1730
16. Catapano, G. European Journal of Heart Failure 10 (2008) 428-434
17. Erbs, S. Am Heart J 2003;146:280-5.
18. Piccirillo, G. Hypertension. 2003; 41:1240-1245
19. Hanninen, S. J Am Coll Cardiol 2006;47:354-61
20. Keith, M. J Am Diet Assoc. 2009;109:1406-1410
21. Le Bouil, A. Clin Chem 1992 June;38(6):1192-3
22. Alsafwah, S. Clin Med Res 2007; 5(4):238
23. Flores-Mateo, G. Am J Clin Nutr 2006;84:762-73
24. Pilz, S. Br J Nutr. 2009 May;101(10):1534-4
25. Lee, J. Omega-3 Fatty Acids for Cardioprotection. Mayo Clin Proc, 2008; 83 (3): 324-332
26. GISSI investigators, Lancet 2008; 372: 1223-30
27. Fish Consumption, Fish Oil, Omega-3 Fatty Acids, and Cardiovascular Disease—American Heart Association Scientific Statement

While the invention has been described in detail and by reference to specific examples and embodiments, those skilled in the art will recognize that deviations and variations are possible without departing from the scope of the invention as defined by the following claims.

What is claimed is:

1. A nutritional supplement comprising the following approximate amounts:

| Vitamin/mineral | Range | |
|---|---|---|
| Vitamin C | 60-150 | mg |
| Vitamin D (as cholecalciferol) | 400-10,000 | IU |
| Thiamin (Vitamin B1) | 1-50 | mg |
| Riboflavin (Vitamin B2) | 1-10 | mg |
| Vitamin B6 | 2-50 | mg |
| Folic Acid | 0.4-5 | mg |

2. The supplement of claim 1 wherein the supplement is further characterized in that it does not contain amounts of Vitamin A, Vitamin K, Vitamin E, or copper.

3. The supplement of claim 2 wherein the supplement further comprises omega fatty acids in an amount up to about 4000 mg (calculated based on the amount of DHA+EPA).

4. The supplement of claim 3 wherein the amount of the omega fatty acids is about 850 mg to 1000 mg.

5. The supplement of claim 1 wherein the amount of Vitamin A is up to about 0.7 mg RE.

6. The supplement of claim 1 wherein the supplement contains about 2 to 10 mg Vitamin B6.

7. The supplement of claim 1 wherein the supplement contains about 1 to 10 mg Vitamin B 1.

8. The supplement of claim 1 wherein the supplement contains about 1 to 4 mg Vitamin B2.

9. The supplement of claim 1 wherein the supplement contains about 400 to 1000 IU of Vitamin D.

10. The supplement of claim 8 wherein the supplement contains about 800 to 1000 IU of Vitamin D.

11. The supplement of claim 1 containing one or more of the following in the approximate amounts indicated:

| | | |
|---|---|---|
| Vitamin E (as d-alpha tocopherol) | 0-400 | IU |
| Niacin (as Niacinamide) | 0-1000 | mg |
| Vitamin B12 | 0-1000 | mcg |
| Iron | 0-150 | mg |
| Zinc | 0-50 | mg |
| Selenium | 0-400 | mcg |
| Copper | 0-10,000 | mcg |

12. A method for preventing or delaying the progression or complications of cardiovascular disease which comprises administering to the patient on a daily basis the nutritional supplement defined in claim 1.

13. The supplement of claim 3 wherein the supplement contains about 2 to 10 mg Vitamin B6.

14. The supplement of claim 3 wherein the supplement contains about 1 to 10 mg Vitamin B1.

15. The supplement of claim 3 wherein the supplement contains about 1 to 4 mg Vitamin B2.

16. The supplement of claim 3 wherein the supplement contains about 400 to 1000 IU of Vitamin D.

17. The supplement of claim 3 containing one or more of the following in the approximate amounts indicated:

| | | |
|---|---|---|
| Vitamin E (as d-alpha tocopherol) | 0-400 | IU |
| Niacin (as Niacinamide) | 0-1000 | mg |
| Vitamin B12 | 0-1000 | mcg |
| Iron | 0-150 | mg |
| Zinc | 0-50 | mg |
| Selenium | 0-400 | mcg |
| Copper | 0-10,000 | mcg |

18. A method for preventing or delaying the progression or complications of cardiovascular disease which comprises administering to the patient on a daily basis the nutritional supplement defined in claim 3.
